(19) United States
(12) Patent Application Publication
(10) Pub. No.: US 2024/0274290 A1
(43) Pub. Date: Aug. 15, 2024

(54) PREDICTING CHANGES IN RISK BASED ON INTERVENTIONS

(71) Applicant: Waymark, Inc., San Francisco, CA (US)

(72) Inventors: Sanjay Basu, San Francisco, CA (US); Aaron Baum, New Haven, CT (US); Sadiq Patel, Exeter, NH (US)

(21) Appl. No.: 18/108,433

(22) Filed: Feb. 10, 2023

(51) Int. Cl. G16H 50/30; G16H 10/60; G16H 20/00

(52) U.S. Cl. CPC G16H 50/30; G16H 10/60; G16H 20/00

(57) ABSTRACT

Systems and methods for predicting changes in risk based on interventions are presented herein. In an example computer-implemented method, a computing device may receive, from a first source, first information and receive, from a second source, second information. The computing device may generate patient information by linking the first information and the second information using a linkage, corresponding to a patient. The computing device may generate using one or more trained machine learning models, a risk prediction for the patient and a change in risk prediction for the patient corresponding to an intervention. The computing device may output the risk prediction and the change in risk prediction for the patient.

### FIELD

This disclosure relates generally to healthcare modeling, and more specifically to predicting change in risk based on interventions.

### BACKGROUND

Healthcare costs in many cases can be high and yet still not result in good outcomes. For populations with heightened needs, like patients receiving Medicaid benefits, the need for improving outcomes relative to expenditures may be particularly acute. Some approaches to reducing costs and improving outcomes may include modeling the cost of caring for patients at the population level. However, such models may be inaccurate or based on non-representative subsets of the population. Moreover, such models may predict cost for aggregate populations but may lack predictive power for any particular patient.

### SUMMARY

Various examples are described for predicting changes in risk based on interventions. In an example computer-implemented method, a computing device may receive, from a first source, first information. The computing device may also receive, from a second source, second information. The computing device may generate patient information by linking the first information and the second information using a linkage, corresponding to a patient. The computing device may generate, using one or more trained machine learning models, a risk prediction for the patient and a change in risk prediction for the patient corresponding to an intervention. The computing device may output the risk prediction and the change in risk prediction for the patient.

An example system may include one or more processors configured to receive, from a first source, first information and receive, from a second source, second information. The one or more processors may generate patient information by linking the first information and the second information using a linkage, corresponding to a patient. The one or more processors may generate, using one or more trained machine learning models, a risk prediction for the patient and a change in risk prediction for the patient corresponding to an intervention. The one or more processors may output the risk prediction and the change in risk prediction for the patient.

An example non-transitory computer-readable medium may store a set of instructions that include one or more instructions that, when executed by one or more processors of a device, cause the device to receive, from a first source, first information and receive, from a second source, second information. The instructions may include an operation to generate patient information by linking the first information and the second information using a linkage, corresponding to a patient. The instructions may include operations to generate, using one or more trained machine learning models, a risk prediction for the patient and a change in risk prediction for the patient corresponding to an intervention. The instructions may include operations to output the risk prediction and the change in risk prediction for the patient.

These illustrative examples are mentioned not to limit or define the scope of this disclosure, but rather to provide examples to aid understanding thereof. Illustrative examples are discussed in the Detailed Description, which provides further description. Advantages offered by various examples may be further understood by examining this specification.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIG. 1 depicts an example of a system for predicting changes in risk based on interventions.

FIG. 2 depicts an example of a system for predicting changes in risk based on interventions.

FIG. 3 depicts example data sources for predicting changes in risk based on interventions.

FIG. 4 shows an example method for predicting changes in risk based on interventions.

FIG. 5 shows an example computing device suitable for predicting changes in risk based on interventions.

### DETAILED DESCRIPTION

Examples are described herein in the context of predicting changes in risk based on interventions. Those of ordinary skill in the art will realize that the following description is illustrative only and is not intended to be in any way limiting. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

The healthcare system may be ineffective for populations with limited income and resources, including patients receiving benefits from government programs like Medicaid. Healthcare costs may be high and yet still not map to good outcomes. For populations with heightened needs, like patients receiving Medicaid benefits, the need for improving outcomes relative to expenditures is particularly acute. Some approaches to meeting the special needs of Medicaid patients may include innovative uses of modern technologies. But such technologies can be constrained by workflows that emphasize billing and insurance. Creativity and experimentation in deploying primary care or improvements in access to healthcare may be difficult to promote with existing workflows.

Some approaches to reducing cost and improving outcomes may include modeling the cost of caring for patients at the population level. However, such models may be inaccurate or based on non-representative subsets of the population. For example, some models may lack accuracy in long-tail probability distributions that can correspond to Medicaid populations. The available Medicaid data may be of poor quality, relative to Medicare or private insurance data. Moreover, such models may predict cost for aggregate populations but may lack predictive power for any particular patient. Certain aspects of the present disclosure aim to deliver correctly timed interventions for particular patients.

Some approaches to reducing cost and improving outcomes may include machine learning models. For example, some machine learning models may include recommendation or steerage models, which may be trained to suggest the best action to take at a given time. Such models are usually ineffective when trained only using traditional electronic health records or insurance claims data. Health records and insurance claims are created for financial reimbursement purposes. Machine learning models trained using such data may not be able to identify which intervention will be most effective for a particular patient, whether that patient will benefit from that intervention, and which modality for carrying out the intervention will yield the best results.

Certain aspects of the present disclosure provide systems and methods to enrich traditional medical records and insurance claims data with new information generated from patient engagements such as details about social relationships, different forms of patient outreach, patient outcomes, and other social determinants of health. Such information may enhance the ability of providers to identify “rising risk” patients who are not yet “high-cost claimants” but are on the pathway to potentially experiencing healthcare catastrophes. Disclosed methods may improve on predicting the risk of negative outcomes for a population to predicting which intervention is most beneficial for a particular patient. For example, a young Medicaid patient recently diagnosed with poorly controlled diabetes may be a candidate for a variety of low-cost interventions with significant benefits that may not be reflected in a model that predicts the probability of a bad outcome for a cohort of comparable patients.

Systems and methods for predicting changes in risk based on interventions are presented herein. For example, in one computer-implemented method, a computing device for predicting changes in risk based on interventions may receive data from one or more external data sources. The computing device may be a healthcare data manager or other suitable component for predicting changes in risk based on interventions. The external data sources may include insurance claims data and electronic health records, among others. The computing device may generate information about a patient by linking the data from the external sources. For example, data from a first source and a second source may be combined using unique identifiers common to both sources, like a full name or social security number. The computing device may use a trained machine learning model to create a risk prediction for the patient. For example, the risk prediction may include an expected cost of treatment, a probability of recovery, or a likelihood of a negative outcome. The computing device may also generate a prediction of the change in risk for particular interventions. For example, for a patient with diabetes, the change in risk prediction may include a reduction in the expected cost of treatment for a course of insulin or an increase in the probability of recovery for a particular lifestyle change. Interventions may include the time and location of the proposed interventions, among other properties. The computing device may output both the risk prediction and the change in risk prediction for particular interventions. For example, the predictions may be output to a customer relationship management (“CRM”) system for use in managing treatment for the patient.

In some examples, the computing device may receive additional data including patient engagement data. The patient engagement data may be generated by a healthcare provider using a healthcare CRM. For example, the healthcare provider may gather patient engagement data by interviewing patients. The patient engagement data may include patient narratives, relationships, and social determinants of health, among other possibilities. The patient engagement data may be linked with the data from external sources. The patient engagement data may be used to identify interventions that reduce the predicted risk in terms of cost or likelihood of recovery. For example, patient engagement data may be used to identify a urinary tract infection prior to a costly emergency room visit or to recommend a mammogram at an age appropriate to reduce the risk of undiagnosed cancer.

In some examples, the trained machine learning model may be an ensemble model that includes one or more machine learning models. For example, the trained machine learning model may include any combination of neural networks, gradient boosted machines, random forests, generalized linear models, and recommendation or steerage models. Other machine learning models may also be combined. The trained machine learning model may be used to generate a score. The score may correspond to a likelihood of success associated with a particular intervention. For example, a high score may indicate a significant expected reduction in cost or increase in the probability of recovery. The score may be composed using the outputs of the one or more machine learning models according to weights assigned to the models. The score may be provided to applications that can use the score to, for example, rank or sort interventions. The applications may include the healthcare CRM that can be used by healthcare providers to select interventions for particular patients.

In one example of a healthcare provider selecting a scored intervention using the healthcare CRM, the healthcare provider using the healthcare CRM may determine that a high score associated with an intervention including a prescription drug treatment fails to correspond with an expected outcome. In such a case, the healthcare provider may provide a correction associated with an outcome of the intervention. The healthcare provider can provide a correction to the computing device that includes an indication that the intervention failed to correspond with the expected outcome. The computing device can then update the weights associated with the one or more machine learning models in order to achieve a lower score for that intervention. Such feedback may happen in real time based on a particular patient or may happen based on outcomes across a number of patients.

In some examples, the computing device may receive feedback associated with the patient. For example, a healthcare provider may determine the outcome or cost of a particular intervention. The feedback may contain data associated with the intervention including, for example, medications included with the intervention or data from hospital visits resulting from the intervention. The healthcare provider may use a healthcare CRM to generate and provide the feedback to the computing device. The computing device may process the feedback and generate patient information, including identification of one or more predictor variables and one or more outcome variables. The processed patient information may be used, with the data from external sources and/or the patient engagement data, to further train the trained machine learning model. For example, the feedback may be used to continuously update and train the machine learning model while it is operating.

This illustrative example is given to introduce the reader to the general subject matter discussed herein and the disclosure is not limited to this example. The following sections describe various additional non-limiting examples and examples of predicting changes in risk based on interventions.

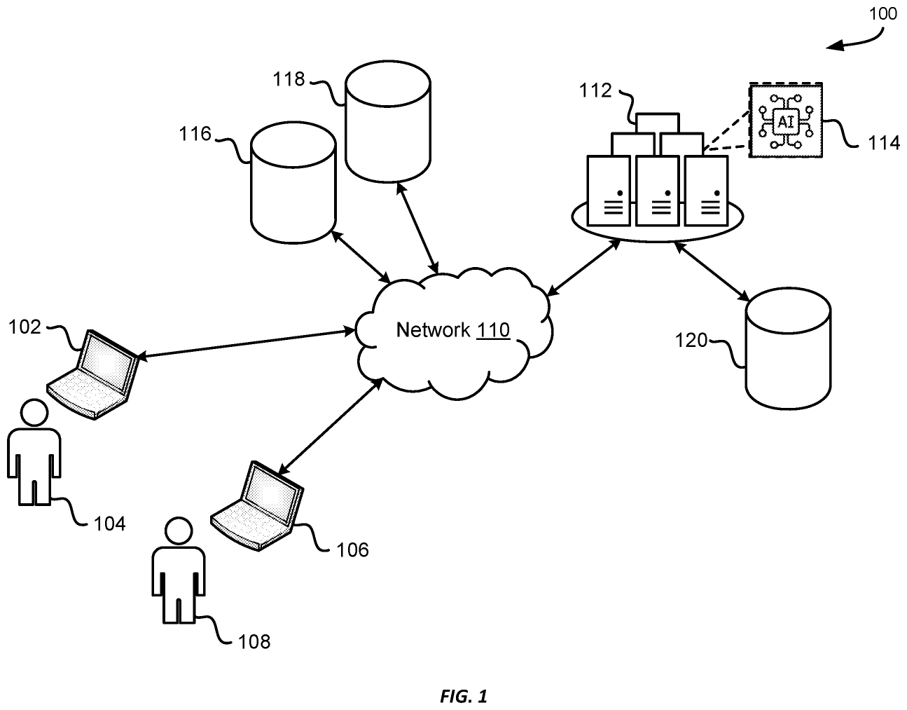

Referring now to FIG. 1, FIG. 1 shows an example system 100 for predicting changes in risk based on interventions. The example system includes two client devices 102, 106 and a remote cloud server system 112. These components are communicatively connected to each other via one or more intervening networks, collectively illustrated as network 110. The intervening networks may include the internet or any other suitable networks that may include any local area network (“LAN”), metro area network (“MAN”), wide area network (“WAN”), cellular network (e.g., 3G, 4G, 4G LTE, 5G, etc.), or any combination of these.

The cloud server system 112 includes one or more server computers located remotely from the client devices 102, 106. The cloud server system 112 may include a healthcare data manager that executes software to predict risk and changes in risk for particular interventions for specific patients. To do this, the cloud server system 112 maintains one or more machine learning models 114 that have been trained to predict risk and changes in risk for particular interventions, as will be discussed in greater detail below. The machine learning models 114 may be an ensemble of machine learning models whose outputs are combined to produce a composite score. The composite score may be calculated according to weights on the models making up the machine learning models 114, such that some models make greater or lesser contributions to the composite score.

The cloud server system 112 receives data from one or more external data sources 116, 118. The external data sources 116, 118 may include data from a variety of sources external to the cloud server system 112. The external data sources 116, 118 may include, for example, public health records, data from academic research, or insurance claims data, among many others. The external data sources 116, 118 can also include data collected by the healthcare data manager and stored, for example, in a cloud storage provider. The cloud server system 112 also receives data from one or more internal data sources 120. For example, the internal data sources may include patient engagement data collected by a healthcare provider 108 using a client device 106 that is subsequently provided to the cloud server system 112, as will be discussed below.

One of the two depicted client devices 106 is executing healthcare customer relationship management (“CRM”) software. Healthcare CRM software may be used to plan and manage patient care. The client device 106 is used by a healthcare provider 108 to, for example, view patient lists, manage patient care, and evaluate and select interventions and tasks. The client device 106 interacts with the cloud server system 112 to, for example, receive data including interventions for specific patients and provide feedback on the recommended interventions. The healthcare CRM software can also receive communications from patients, other health providers, or the cloud server system 112, such as text or voice messages, as well as other information about patient health and care.

The other depicted client device 102 is used by an administrator of the healthcare data manager 104 to receive feedback from the other client device 106 and to monitor the output of the machine learning models 114. The administrator of the healthcare data manager 104 reviews the feedback and adjusts the weightings on the models making up the machine learning models 114 in accordance with the feedback received from the client device 106. The administrator of the healthcare data manager 104 may also review feedback from the client device 106 and add additional predictor and outcome variables to the one or more machine learning models 114 according to the feedback. The client device 102 also allows the administrator of the healthcare data manager 104 to communicate with the healthcare provider 108, such as to text message or have voice or video calls with the healthcare provider 108 in order to assist with interpretation of the feedback.

The client devices 102, 106 may be any suitable client device for the respective user, including handheld devices (such as smartphones and tablets), portable devices (such as laptop computers), or desktop computers. In some examples the client devices 102, 106 may execute client software via a web browser by accessing a link (e.g., a uniform resource locator or “URL”) to a web application hosted by the cloud server system 112. In other examples, the client software for one or both client devices 102, 106 may be locally executed. And while this example system 100 shows only two client devices 102, 106, any number of client devices may be included in example systems according to this disclosure. Further, it should be appreciated that the client devices 102, 106 may be remote from each other. For example, the healthcare provider 108 may have their client device 106 at their home, while the administrator of the healthcare data manager 104 may have their client device 102 at their office.

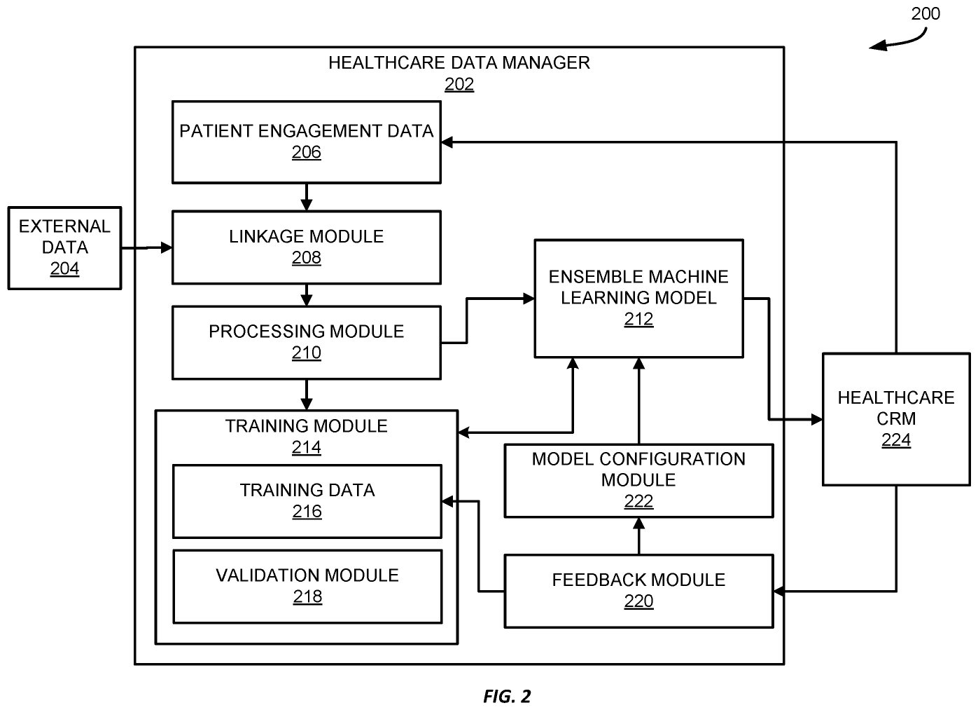

Referring now to FIG. 2, FIG. 2 depicts an example of a system 200 for predicting changes in risk based on interventions. The system 200 includes a healthcare data manager 202. The healthcare data manager 202 may be associated with one or more healthcare providers. The healthcare data manager 202 may be a standalone server, multiple servers connected over a network, one or more virtual machines running a cloud provider, or other suitable configuration.

The healthcare data manager 202 receives data from various sources. The data may be used as input to an ensemble machine learning model 212 or to train the ensemble machine learning model 212. The data may include external data 204 or patient engagement data 206. The external data 204 may include data from a variety of sources external to the healthcare data manager 202. The patient engagement data 206 includes data collected by a healthcare provider that is subsequently provided to the healthcare data manager 202. For example, the patient engagement data 206 may include data generated by and provided by a healthcare provider using a healthcare CRM 224.

The external data 204 and the patient engagement data 206, as well as any other sources of data, are combined by a linkage module 208. The linkage module 208 may aggregate and process data from one or more data sources to generate data that is keyed to individual patients via a linkage. The linkage may be a subset of the data that can be used to join data from disparate sources. For example, data from a first source and a second source may be combined using unique identifiers common to both sources, like a full name or social security number. In some examples, some sources of external data 204 may include events that are uniquely associated with an individual patient. Likewise, the patient engagement data 206 may include details associated with individual patients. Some sources of external data 204, however, may include data associated with populations, regions, symptoms, conditions, periods of time, etc. that are not linked directly to individual patients.

The linkage module 208 can combine disparate data sources into a format that is suitable for consumption by or training of the ensemble machine learning model 212. In some examples, the linked data may include time-ordered or time-series data for individual patients. In other words, the linked data may include data structures associated with individual patients at particular times. The linked data may include a plurality of data structures for individual patients at different times. For example, linked data may include rows in a relational database. Rows may correspond to individual patients' status at a particular moment in time, such as based on test results or assessment data. Rows may contain data linked from the external data 204 and the patient engagement data 206, that corresponds to the particular moment in time. For example, the row may include linked data that indicates that an individual patient was admitted to the hospital on a particular date, a measurement of air pollution on that date, and the probability of occurrence of a medical condition for patients with comparable demographic characteristics on that date. The use of linked data as input to and as a source of training data for the ensemble machine learning model 212 may allow for the prediction of both risk, including healthcare costs, as well as the prediction of change in risk for specific interventions for individual patients.

The healthcare data manager 202 includes a processing module 210. The processing module 210 may define predictor variables and outcome variables. The predictor variables may correspond to machine learning features and outcome variables may correspond to machine learning examples. The processing module 210 may receive the linked data from the linkage module 208 and identify candidate data structures for definition as predictor variables and outcome variables. In some examples, the processing module 210 may define predictor variables and outcome variables automatically according to predefined criteria. In some examples, the predictor variables and outcome variables may be identified or defined manually by users of the healthcare data manager 202. Predictor variables may include, among others, data on demographics, social determinants of health, prior claims history, diagnoses, medications, provider characteristics, residential area-level characteristics, and outreaches, engagements, and interventions by healthcare providers. In some examples, predictor variables may include feedback from users of applications consuming the output of the ensemble machine learning model 212. For example, healthcare providers may use the healthcare CRM 224 and provide feedback relating to the predictions made by the ensemble machine learning model 212. The feedback may be utilized as a predictor variable. The outcome variables may include, among others, data on ambulatory care-sensitive emergency room visits, hospitalizations, engagements with healthcare providers, medical and pharmaceutical costs, patient goal fulfillment rates, care gap closure rates by the National Committee for Quality Assurance (“NCQA”) Healthcare Effectiveness Data and Information Set (“HEDIS”), and Medicaid disenrollment.

The ensemble machine learning model 212 receives input from the processing module 210. The ensemble machine learning model 212 may be a trained machine learning model or it may be trained using training data 216. The ensemble machine learning model 212 may be continuously trained using feedback from, for example, a healthcare CRM 224. The machine learning module may include an “ensemble” or “orchestra” of machine learning models trained using the same or subsets of the same training data 216. The machine learning models making up the ensemble machine learning model 212 may include one or more machine learning models. For example, the one or more machine learning models making up the ensemble machine learning model 212 may include neural networks, gradient boosted machines, random forests, generalized linear models, and recommendation or steerage models.

Any suitable machine learning model may be used according to different examples, such as deep convolutional neural networks (“CNNs”); a residual neural network (“Resnet”), or a recurrent neural network, e.g. long short-term memory (“LSTM”) models or gated recurrent units (“GRUs”) models, a three-dimensional CNN (“3DCNN”), a dynamic time warping (“DTW”) technique, a hidden Markov model (“HMM”), a support vector machine (SVM), decision tree, random forest, etc., or combinations of one or more of such techniques—e.g., CNN-HMM or MCNN (Multi-Scale Convolutional Neural Network). Further, some examples may employ adversarial networks, such as generative adversarial networks (“GANs”), or may employ autoencoders (“AEs”) in conjunction with machine learning models, such as AEGANs or variational AEGANs (“VAEGANs”).

Example machine learning models making up the ensemble machine learning model 212 may have specific advantages with respect to predicting change in risk based on interventions. For example, neural networks may be used to model complex interactions among predictor variables that cannot otherwise be explained. Gradient boosting machines can be more accurate, can learn in a non-linear fashion, can improve over time, and can be well-suited to datasets with missing values as with, for example, Medicaid data. Random forest models may work well with small datasets and a minimal amount of configuration. Recommendation or steerage models may be well-suited to ranking potential interventions according to historical patient data.

The ensemble machine learning model 212 may include one or more types of machine learning models to obtain better predictive performance than could be obtained using any one specific kind. The machine learning model ensemble may include a model stacking algorithm. A stacking algorithm may combine or “stack” a plurality of trained machine learning models using a top-level machine learning model which may itself be trained to combine the included machine learning models according to an algorithm to achieve a specified goal. For example, the machine learning models may be combined by normalizing and weighting the contributions from the constituent models. The top-level model may be trained to adjust the weights to achieve the specified learning goal. In some examples, the machine learning model ensemble may operate according to a weighted voting process among the constituent models. Other ensemble approaches may be used including a Bayesian optimal classifier, bootstrap aggregating or “bagging,” boosting, Bayesian model averaging, Bayesian model combination, a bucket of models, or other approach.

The ensemble machine learning model 212 may output a predicted risk for a given patient. For example, the predicted risk may include the expected cost in the absence of any intervention, the expected average cost for patients in a comparable cohort, the average probability of a negative outcome, or other measures of risk. The ensemble machine learning model 212 may also output a prediction of the change in risk for particular interventions. For example, the prediction of the change in risk may include a reduction in cost associated with an intervention for a particular patient, an increase in the probability of recovery associated with an intervention for a particular patient, or other measures of change in risk. The ensemble machine learning model 212 may also output recommendations for patient priority, outreach approach, and intervention type. Patient priority may include an indication of which patients are at the highest risk for negative or expensive outcomes. Outreach approach may include an indication of which modality of contacting or interacting with patients have the highest probability of success. Intervention type may include a specific action to recommend that a patient or healthcare provider perform. A proposed intervention may include the patient that is the target of the intervention, a date and time the intervention can occur, a healthcare provider who will perform the intervention, a modality of the intervention, and a location at which the intervention may occur. For example, recommended interventions for a patient suffering from hunger may include hospitalization by a healthcare provider, an email recommending enrollment in a meal delivery program, or a text message containing a recommendation for a food pantry.

In some examples, the models composing the ensemble machine learning model 212 may be based on the geographic location of a subset of patients served by the healthcare data manager 202. For example, because factors that significantly affect healthcare in one state may differ from the factors that significantly affect healthcare in another state or other localities, such as cities, towns, neighborhoods, or regions, machine learning models may be separately trained to correspond to patients residing or living in those states or localities. In that example, risk predictions and change in risk predictions for patients in a first state may be generated by a machine learning model trained using data relevant for healthcare decisions in the first state, whereas predictions for patients in a second state may be generated by a machine learning model trained using data relevant for healthcare decisions in the second state. The machine learning models corresponding to different geographic locations may share data and they may use data that is applicable to patients at specific locations. The machine learning models may be grouped according to other criteria. For example, machine learning models could be trained that are specific to individual patients, regions, demographics, or other categorizations.

The ensemble machine learning model 212 may output a score corresponding to the recommendations for patient priority, outreach approach, and intervention type. For example, a high score may correspond to a high likelihood of a positive outcome or a significant reduction in cost, given a particular intervention for a particular patient. In some examples, the score may be provided to applications that may then rank the recommendations of the ensemble machine learning model 212 according to the score. The score may include one or more portions. The portions may be weighted contributions from the various machine learning models included in the ensemble machine learning model 212. For example, in an ensemble machine learning model 212 that includes a neural network and a recommendation or steerage model, the output of the neural network may contribute to 25% of the score and the recommendation or steerage model may contribute to 75% of the score. Appropriate normalization methods may be employed to quantify and/or scale the outputs of the various machine learning models prior to summing contributions to the score. In some examples, the outputs of the machine learning models may be combined according to an ensemble machine learning algorithm, including, for example, stacking, bagging, or boosting.

The outputs of the ensemble machine learning model 212 are sent to one or more applications including, for example, a healthcare CRM 224. The outputs of the ensemble machine learning model 212 may be provided by way of an exposed application programming interface (“API”) or other suitable mechanism. The healthcare CRM 224 may display the outputs of the ensemble machine learning model 212, for example, using a suitable graphical user interface (“GUI”). For example, the healthcare CRM 224 may display a ranked list of interventions for a particular patient according to the score, expected cost, or other method for sorting the interventions. The healthcare CRM 224 is only an example client of the healthcare data manager 202. It should be stressed that other types of client applications may receive output from the ensemble machine learning model 212. For example, the output may be exposed over a public API which may be used in a variety of custom applications.

The training module 214 may initially train the ensemble machine learning model 212 using the training data 216 using any suitable supervised, semi-supervised, or unsupervised training technique. The training data 216 may include linked data from the linkage module 208 that has been labeled in the processing module 210. The training data 216 may include data from both the external data 204 and the patient engagement data 206. The training data 216 may be continuously updated via the feedback module 220, which may receive feedback from, for example, the healthcare CRM 224.

The training module 214 receives processed data from the processing module 210. The training module 214 may designate subsets of the processed data as training data 216, validation data, and testing data. The validation data may be used by the validation module 218 to validate the outputs of the ensemble machine learning model 212 after it has been trained. This may ensure that the resulting model is not unduly influenced by the particular characteristics of the training data 216. The testing data may be used by the validation module 218 to again validate the outputs of the ensemble machine learning model 212 to confirm that the ensemble machine learning model 212 is properly trained. The training data 216, the validation data, and the test data may be periodically updated or replaced to prevent the ensemble machine learning model 212 from overfitting the data or other potential problems.

The validation module 218 may also compare the output of the ensemble machine learning model 212 with pre-trained models. For example, the pre-trained models may include commercially available models that may be used to evaluate the performance of the one or more machine learning models making up the ensemble machine learning model 212. In some examples, a pre-trained model may generate a risk prediction. The validation module 218 can compare the risk prediction made by the pre-trained model with the risk prediction made by the one or more machine learning models to validate the performance of the one or more machine learning models or of the ensemble machine learning model 212.

The feedback module 220 receives feedback from external sources and provides the feedback to the training module 214 or to the model configuration module 222. For example, the feedback module 220 may receive feedback from a healthcare CRM 224 on the interventions corresponding to the ensemble machine learning model 212 output according to factors including appropriateness, interpretability, relevance, missing elements, overemphasis, as well as other considerations. The feedback may be added to the training data 216. The ensemble machine learning model 212 may be continuously trained as new data is added to the training data 216 via the feedback module 220. The feedback module 220 may also add data to the patient engagement data 206, which may then be input to the trained machine learning model. In some examples, the feedback may be used as a predictor variable. For instance, feedback from a healthcare provider may be based on clinical expertise that is not otherwise captured in the external data 204 or patient engagement data 206. The feedback may include recommendations or evaluations of interventions that may implicitly include the clinical expertise and may thus be used as predictor variables during offline or online training of the ensemble machine learning model 212. For example, healthcare providers using the healthcare CRM 224 may provide feedback on a given intervention associated with a score or change in risk prediction by rating the interpretability and quality of the intervention using a scale of 1 to 5 using a GUI provided by the healthcare CRM 224.

The feedback module 220 provides feedback to the model configuration module 222. The model configuration module 222 also receives input from clients of the healthcare data manager 202 that correspond to the accuracy and usefulness of the outputs of the ensemble machine learning model 212. For example, a user of the healthcare data manager 202 may determine that a ranked list of interventions for a given patient fails to sufficiently account for the socio-economic status of the patient. The user may determine that the ranking is significantly due to a contribution from a particular neural network. The user may input updated weightings of the machine learning models to the model configuration module 222 to reduce the contribution from the particular neural network. The model configuration module 222 may update the weights of the machine learning models making up the score that is output from the ensemble machine learning model 212 such that a smaller portion of the score is derived from the particular neural network. The ensemble machine learning model 212 may then output a score that includes a smaller proportional contribution from the particular neural network. In some examples, the input to the model configuration module 222 can provide input to the algorithm combining the models in the ensemble machine learning model 212. The ensemble algorithm may then update the relative importance of the constituent machine learning models according to the output of the ensemble algorithm.

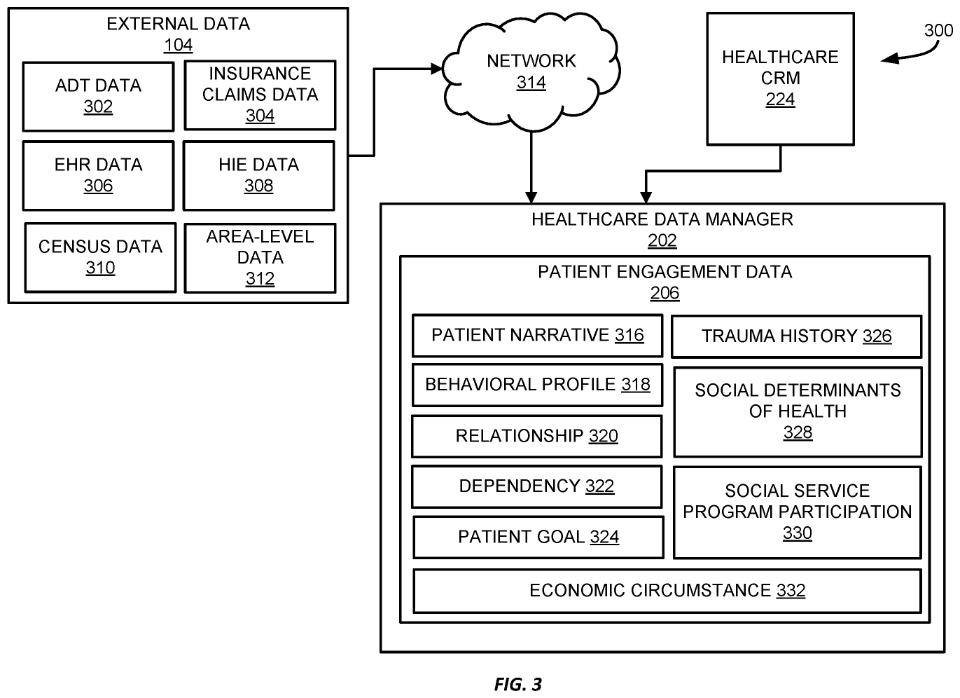

Referring now to FIG. 3, FIG. 3 depicts example data sources 300 for predicting changes in risk based on interventions. The healthcare data manager 202 may receive data from various sources. The data may be used as input to an ensemble machine learning model 212 or to train the ensemble machine learning model 212. The data may include external data 204 or patient engagement data 206.

The external data 204 may include data from a variety of sources external to the healthcare data manager 202. The external data 204 maybe received by the healthcare data manager 202 through one or more networks, represented by network 314, which may include one or more public or private networks, including the internet.

The external data 204 may include admit, discharge, and transfer (“ADT”) data 302. The ADT data 302 may be received in real-time, as the data is generated by an external source. The ADT data 302 may include real-time data indicating that patients have been admitted, discharged, or transferred from an emergency room or hospital. The ADT data 302 may be particularly relevant for identifying interventions that may have been expensive relative to the resultant outcome. The external data 204 may include insurance claims data 304. The insurance claims data 304 may include pharmaceutical and medical insurance claims. For example, the insurance claims data 304 may include Medicaid-specific claims of large cohort of the population.

The external data 304 may also include electronic health records (“EHR”) data 306. The EHR data 306 may include a patient's medical history, diagnoses, medications, treatment plans, immunization dates, allergies, radiology images, and laboratory and test results as well as billing data including cost and insurance coding metadata. The external data 204 may include health information exchange (“HIE”) data 308. HIE data broadly refers to the transmission of healthcare data among medical facilities, providers, and patients electronically and may include medical history, medications, laboratory results, progress notes, referral data, or discharge summaries, among other data.

In some examples, the external data 204 can include census data 310. Census data 310 may include, for example, publicly available data from state and national censuses. The external data 204 may include area-level data 312. Area-level data 312 may include data applicable to individuals living or residing in particular geographic bounds. For example, the area-level data 312 may include air pollution data for a state, cancer statistics for a region, or water availability for a county, among other possibilities. It should be stressed that these data sources making up the external data 204 are just examples. The external data 204 may include additional sources of data from any external source, including sources that can be linked to individual patient data using a linkage. The linkage may be a unique identifier like a name or social security number, a geographic location, patient data, or any other data that may be used to link patient data to data from an external source.

The patient engagement data 206 generally includes patient-specific data obtained from any of various possible sources, including data collected by healthcare providers, social workers, surveys or assessments, or from patients directly. The patient engagement data 206 may include data collected by the healthcare provider that is subsequently provided to the healthcare data manager 202. For example, the patient engagement data 206 may include data generated by or provided by the healthcare CRM 224. The patient engagement data 206 may be stored within the healthcare data manager 202 as shown, but other examples are possible. For example, the patient engagement data 206 could be stored in the healthcare CRM 224 and provided to the healthcare data manager 202 via an API.

The patient engagement data 206 may also include a patient narrative 316. For example, a healthcare provider using the healthcare CRM 224 may obtain a patient narrative 316 from a patient while providing care or during a routine screening. The patient narrative 316 may include information on any subject relevant to the prediction of risk or change in risk for a given intervention. The patent narrative 316 may be in a text or audio format. Natural language processing (“NLP”) or other model may be used to convert the patient narrative 316 into a machine-readable format suitable for input to the ensemble machine learning model 212.

Some examples of patient engagement data 206 can include a behavioral profile 318. For example, a healthcare provider using the healthcare CRM 224 may generate a behavioral profile 318 of a patient while providing care or during a routine screening by the healthcare provider. The behavioral profile 318 may include a summary of the behavior patterns of a patient as observed by or told to a healthcare provider. The behavioral profile 318 may be provided in a standardized format, a narrative format, or other format suitable for input to the ensemble machine learning model 212.

A relationship 320 may be included in the patient engagement data 206. For example, a healthcare provider using the healthcare CRM 224 may generate a relationship 320 of a patient while providing care or during a routine screening by the healthcare provider. The relationship 320 may be represented by a social graph, including interconnected nodes that represent the relationship 320, using text, or other suitable format for input to the ensemble machine learning model 212. The relationship 320 may include relationships among patients, providers, family members, or other individuals relevant to the prediction of risk or change in risk for a given intervention.

Included in the patient engagement data 206 may be a dependency 322. For example, a healthcare provider using the healthcare CRM 224 may generate the dependency 322 of a patient while providing care or during a routine screening. The dependency 322 may include the amount of care, the complexity of care, or the amount of time needed for care for a particular patient. The dependency 322 may include pharmaceutical dependencies or other chemical dependencies or lifestyle dependencies. The dependency 322 may be represented in a narrative format, including time-series data illustrating, for example, how the dependency 322 has changed over time. Or the dependency 322 may be represented in any suitable format for input into the ensemble machine learning model 212.

The patient engagement data 206 can also include a patient goal 324. For example, a healthcare provider using the healthcare CRM 224 may generate the patient goal 324 while providing care or during a routine screening. For example, the patient goal 324 may include an objective corresponding to a proposed intervention. A patient goal 324 may include recovery, a degree of recovery, or a particular outcome. The patient goal 324 may be represented in a narrative or other textual format, encoded according to a standardized format, or any other suitable format for input to the ensemble machine learning model 212.

In some examples, the patient engagement data 206 may include a trauma history 326. For example, a healthcare provider using the healthcare CRM 224 may generate the trauma history 326 while providing care or during a routine screening. The trauma history 326 may include information concerning past patient traumas relevant to the prediction of risk or change in risk for a given intervention. The trauma history 326 may be in a text or audio format. Natural language processing (“NLP”) or other model may be used to convert the trauma history 326 into a machine-readable format suitable for input to the ensemble machine learning model 212. The trauma history 326 may include time-series data including a series of past events.

One or more social determinants of health 328 can be included in the patient engagement data 206. For example, a healthcare provider using the healthcare CRM 224 may obtain the social determinants of health 328 while providing care or during a routine screening. The social determinants of health 328 may include the conditions in the environments where patients live that may affect health, functioning, outcomes, and risks. The social determinants of health 328 may include data relating to economic stability, access to education, quality of education, access to health care, quality of healthcare, residential and work environments, social context, access to food, community context, and other factors. The social determinants of health 328 may include time-series data indicating how the social determinants of health 328 have changed over time. The social determinants of health 328 may be narrative form, standardized form, or other format suitable for input to the ensemble machine learning model 212.

Social service program participation 330 may be included in some examples of the patient engagement data 206. For example, a healthcare provider using the healthcare CRM 224 may determine social service program participation 330 while providing care or during a routine screening. The social service program participation 330 may include data concerning a patient's use of social services including temporary financial assistance programs, supplemental nutrition assistance programs, educational programs, childcare programs, foster care programs, adoption programs, senior assistance programs, homelessness programs, veteran support programs, among others. The social service program participation 330 may include time-series data indicating how the social service program participation 330 has changed over time. The social service program participation 330 may be in a narrative format, standardized format, or other format suitable for input to the ensemble machine learning model 212.

The patient engagement data 206 may also include an economic circumstance 332. For example, a healthcare provider using the healthcare CRM 224 may determine the economic circumstance 332 while providing care or during a routine screening. The economic circumstance 332 may include details relating to the patient's financial status, ability to pay, tax records, hardships, or other factors. The economic circumstance 332 may include time-series data indicating how the economic circumstance 332 has changed over time. The economic circumstance 332 may be in a narrative format, standardized format, or other format suitable for input to the ensemble machine learning model 212.

It should be stressed that these data sources making up the patient engagement data 206 are just examples. The patient engagement data 206 may include additional sources of data from any source designated by users of the healthcare data manager 202. For example, the patient engagement data 206 may include text or SMS messages sent to healthcare providers from patients or other examples of freeform text. In that example, NLP or other technologies may be used to translate the freeform text into a form suitable for input to the ensemble machine learning model 212.

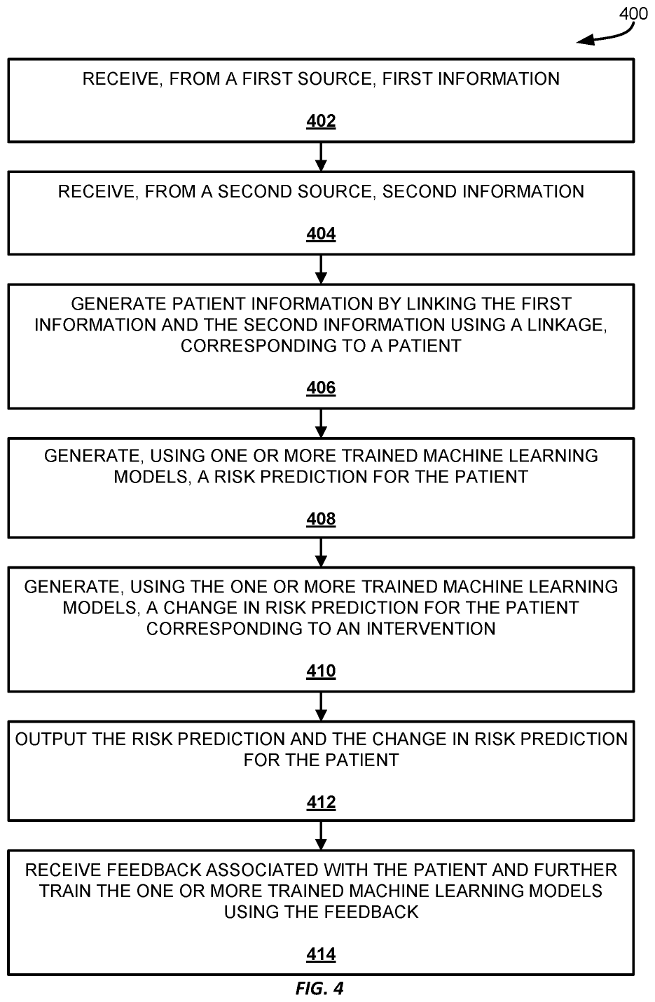

Referring now to FIG. 4, FIG. 4 shows an example method 400 for predicting changes in risk based on interventions. These methods can be implemented by the healthcare data manager 202 of system 200, or any other suitable component. These methods can be read with reference to the examples of FIGS. 1-3 for illustrative purposes. It should be appreciated that this example method 400 provides a particular method for predicting changes in risk based on interventions. Other sequences of operations may also be performed according to alternative examples. For example, alternative examples of the present invention may perform the steps outlined above in a different order. Moreover, the individual operations illustrated by these methods may include multiple sub-operations that may be performed in various sequences as appropriate to the individual operation. Furthermore, additional operations may be added or removed depending on the particular applications. One of ordinary skill in the art would recognize many variations, modifications, and alternatives.

Turning first to block 402, in block 402 a healthcare data manager 202 may include a computing device that may receive, from a first source, first information. For example, the computing device may receive data from any available data source, which may include the data sources, e.g., external data 204 and patient engagement data 206, discussed above with respect to FIG. 2. However, data from other data sources may be received as well, such as from manual input or patient surveys or assessments provided by third parties, among other possibilities. The external data 204 or patient engagement data 206 may include first information. The first information may be received by the computing device in a variety of formats that may be processed by the healthcare data manager 202 into a format suitable for input to or training of the ensemble machine learning model 212. The first information may be pushed to the healthcare data manager 202, for example, using an API exposed by the healthcare data manager 202. Alternatively, the first information may be obtained by the healthcare data manager 202 over a network 314 using publicly available APIs, web scraping, downloading, manually uploading, or other suitable method for data ingress.

In block 404, the computing device may receive, from a second source, second information. As in block 402, the computing device may receive external data 204 or patient engagement data 206. The external data 204 or patient engagement data 206 may include second information. The second information may be from the same source or from a different source as the first information.

In block 406, the computing device may generate patient information by linking the first information and the second information using a linkage, corresponding to a patient. For example, information from the external data 204 and the patient engagement data 206 may be combined by a linkage module 208. The linkage module 208 may aggregate and process data from one or more data sources to generate data that is keyed to individual patients using a linkage. The linkage may be a subset of the data that can be used to join data from disparate sources. For example, data from a first source and a second source may be combined using unique identifiers common to both sources, like a full name or social security number. In some examples, unique identifiers may not be available or may be insufficient to distinguish one patient from another. Composite linkages may be constructed using one or more available data structures from the one or more data sources. For example, a composite linkage may be constructed using both full name, location (e.g., state), and social security number.

The computing device may be configured to link data from disparate sources. For example, the computing device may be configured to determine the linkage according to the source of the data. Alternatively, the computing device may be configured to determine the linkage following receipt of the information according to a suitable algorithm. For example, the computing device may use a regular expression to identify social security numbers or infer patient names from data labels. The computing device may use information concerning standardized data formats to determine the linkage. The computing device may use a pre-trained NLP model to infer data that may be linked, including models such as Bidirectional Encoder Representations from Transformers (“BERT”) and Generative Pre-trained Transformer (“GPT”) 3.

The first information and the second information may be linked in a data structure suitable for input to or training of the ensemble machine learning model 212. In some examples, the linked information may include rows in a relational database or objects in an object-oriented database. For example, rows in a relational database may include available data about a patient, or a row may correspond to a particular moment in time, a date, or a date/time range. In this way, the rows in the relational database may be time-series data relating how the patient data has changed over time. Time-series data may ensure that data is considered longitudinally by the ensemble machine learning model 212, including data outside the context of specific events. In another example, patient data may be linked in a document-based store containing one or more documents for a given patient. For example, a document may contain a labeled, timestamped collection of the data or some subset thereof that has been received about a particular patient. In another example, patient data may be stored in a raw format in a data lake or the like. Data stored in an unstructured, raw format may be stored as it is received, without any alteration or reformatting. In this case, the information may be linked using an index used to label the data that contains a linkage. For example, the index may include the linkage (e.g., patient social security number) as well as information about the time and date corresponding to the data.

In block 408, the computing device may generate, using one or more trained machine learning models constituting the ensemble machine learning model 212, a risk prediction for the patient. The ensemble machine learning model 212 may be trained or may be continuously trained using feedback from, for example, the healthcare CRM 224. Training of the ensemble machine learning model 212 may include the identification of and the defining of one or more predictor variables and one or more outcome variables. The predictor variables may correspond to elements of the linked data and may be used to generate values corresponding to the outcome variables. The risk prediction may be composed of or derived from one or more outcome variable values. For example, the risk prediction may be an estimate of the expected cost of healthcare for a patient or an average probability of recovery. The risk prediction may be constructed from outcome variables that include the likely costs of emergency room visits, hospitalizations, engagements with the healthcare providers, pharmaceutical costs, and other modeled outcomes. In some examples, the risk prediction and change in risk prediction may be generated upon the occurrence of one or more triggers. For example, the risk prediction and change in risk prediction may be generated upon the receipt of data from an external source like ADT data 302, including an ADT data stream, or upon feedback from a healthcare provider using the healthcare CRM 224. In other examples, the risk prediction may be generated upon updates to the ensemble machine learning model 212 following updates to the model from online training from feedback or from the model configuration module 222.

In block 410, the computing device may generate, using the one or more trained machine learning models making up the ensemble machine learning model 212, a change in risk prediction for the patient corresponding to an intervention. The change in risk prediction for the patient corresponding to an intervention may be composed of one or more outcome variable values. For example, the change in risk prediction for the patient corresponding to an intervention may include an estimate of a reduction in the expected cost of healthcare for a patient or an increase in the probability of recovery from a given condition given an intervention. The change in risk prediction for the patient corresponding to an intervention may be constructed from outcome variables that include the likely costs of emergency room visits, hospitalizations, engagements with the healthcare providers, pharmaceutical costs, and other modeled outcomes. The change in risk prediction for the patient corresponding to an intervention may be associated with a score, wherein the magnitude of the score corresponds to improved outcomes. For example, a high score may be associated with a reduction in expected costs or an increase in the probability of recovery.

In block 412, the computing device may output the risk prediction and the change in risk prediction for the patient. The risk prediction and the change in risk prediction for the patient may be output in any suitable way for use in an application. For example, the predictions, including a score, may be provided to a healthcare CRM 224. The healthcare CRM 224 may use the score to provide healthcare providers with a list of interventions that are ranked according to the desirability of outcomes in terms of cost or likelihood of recovery. In some examples, the predictions and score may be exposed using a publicly available API, made available for download, published, or other methods of output.

In block 414, the computing device may receive feedback associated with the patient and further train the one or more machine learning models using the feedback. For example, the computing device may receive feedback from a healthcare CRM 224 on the interventions corresponding to the ensemble machine learning model 212 output according to factors including appropriateness, interpretability, relevance, missing elements, overemphasis, as well as other considerations. In some examples, the feedback may be used as a predictor variable. For instance, feedback may include recommendations or evaluations of interventions that may be used as predictor variables during offline or online training of the ensemble machine learning model 212. The feedback may also include evaluations of interventions or other outcome data that may be used as outcome variables during offline or online training of the ensemble machine learning model 212. The feedback may be added to the training data 216. The ensemble machine learning model 212 may be continuously trained as new data is added to the training data 216. The computing device may also add data to the patient engagement data 206, which may then be input to the trained machine learning model. The ensemble machine learning model 212 can use the added predictor variables along with the existing predictor variables to provide estimations for the new outcome variables.

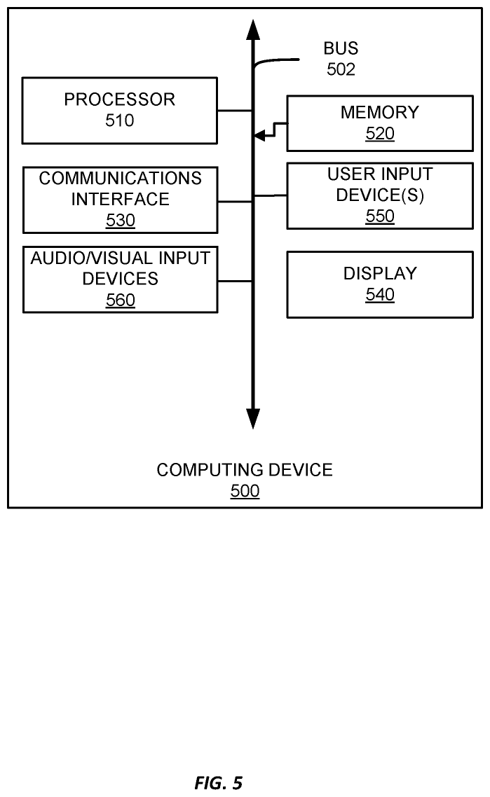

Referring now to FIG. 5, FIG. 5 shows an example computing device 500 suitable for predicting changes in risk based on interventions. The example computing device 500 includes a processor 510 which is in communication with the memory 520 and other components of the computing device 500 using one or more communications buses 502. The processor 510 is configured to execute processor-executable instructions stored in the memory 520 to perform one or more methods for predicting changes in risk based on interventions according to different examples, such as part or all of the example method 400 described above with respect to FIGS. 1-3. The computing device 500, in this example, also includes one or more user input devices 550, such as a keyboard, mouse, touchscreen, microphone, etc., to accept user input. The computing device 500 also includes a display 540 to provide visual output to a user.

The computer device 500 may also include one or more audio/visual input devices 560 to enhance a user's ability to give input to or receive input from a multimedia application or feature, such as a video conference, entertainment application, accessibility features, VR headset, or the like.

The computing device 500 also includes a communications interface 530. In some examples, the communications interface 530 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

While some examples of methods and systems herein are described in terms of software executing on various machines, the methods and systems may also be implemented as specifically-configured hardware, such as field-programmable gate array (FPGA) specifically to execute the various methods according to this disclosure. For example, examples can be implemented in digital electronic circuitry, or in computer hardware, firmware, software, or in a combination thereof. In one example, a device may include a processor or processors. The processor comprises a computer-readable medium, such as a random access memory (RAM) coupled to the processor. The processor executes computer-executable program instructions stored in memory, such as executing one or more computer programs. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example one or more non-transitory computer-readable media, which may store processor-executable instructions that, when executed by the processor, can cause the processor to perform methods according to this disclosure as carried out, or assisted, by a processor. Examples of non-transitory computer-readable medium may include, but are not limited to, an electronic, optical, magnetic, or other storage device capable of providing a processor, such as the processor in a web server, with processor-executable instructions. Other examples of non-transitory computer-readable media include, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may comprise code to carry out methods (or parts of methods) according to this disclosure.

The foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

That which is claimed is:

1. A computer-implemented method, comprising:
receiving, from a first source, first information;
receiving, from a second source, second information;
generating patient information by linking the first information and the second information using a linkage, corresponding to a patient;
generating, using one or more trained machine learning models, a risk prediction for the patient;
generating, using the one or more trained machine learning models, a change in risk prediction for the patient corresponding to an intervention; and
outputting the risk prediction and the change in risk prediction for the patient.

2. The method of claim 1, further comprising receiving, from a third source, third information, wherein the third information includes engagement data from the patient.

3. The method of claim 2, wherein the engagement data comprises a patient narrative and one or more social determinants of health.

4. The method of claim 1, further comprising:
generating, using the one or more trained machine learning models, a score corresponding to the change in risk prediction for the patient, corresponding to the intervention, wherein the magnitude of the score corresponds to a likelihood of success associated with the intervention; and
outputting the score.

5. The method of claim 4, further comprising weighting the one or more trained machine learning models, wherein the score comprises one or more portions corresponding to the one or more trained machine learning models and the portions are proportional to the weight of the corresponding trained machine learning models, and further comprising:
receiving a correction associated with an outcome of the intervention; and
updating the weights of the one or more trained machine learning models based on the correction.

6. The method of claim 1, further comprising:
receiving feedback associated with the patient;
generating first processed patient information by identifying a first predictor variable and a first outcome variable in the patient information;
generating second processed patient information by identifying a second predictor variable and a second outcome variable in the feedback;
generating training data including:
the first information;
the second information;
the first processed patient information; and
the second processed patient information; and
training the one or more trained machine learning models using the training data to predict the first outcome variable and the second outcome variable using the first predictor variable and the second predictor variable.

7. The method of claim 1, wherein the intervention comprises:
the patient that is the target of the intervention;
a date the intervention will occur;
a time the intervention will occur;
a provider who will perform the intervention;
a modality of the intervention; and
a location of the intervention.

8. A system comprising:
one or more processors configured to:
receive, from a first source, first information;
receive, from a second source, second information;
generate patient information by linking the first information and the second information using a linkage, corresponding to a patient;
generate, using one or more trained machine learning models, a risk prediction for the patient;
generate, using the one or more trained machine learning models, a change in risk prediction for the patient corresponding to an intervention; and
output the risk prediction and the change in risk prediction for the patient.

9. The system of claim 8, further comprising receiving, from a third source, third information, wherein the third information includes engagement data from the patient.

10. The system of claim 9, wherein the engagement data comprises a patient narrative and one or more social determinants of health.

11. The system of claim 8, wherein the one or more trained machine learning models comprise a neural network and a recommendation or steerage model.

12. The system of claim 11, further comprising:
generating, using the one or more trained machine learning models, a score corresponding to the change in risk prediction for the patient, corresponding to the intervention, wherein the magnitude of the score corresponds to a likelihood of success associated with the intervention; and
outputting the score.

13. The system of claim 12, further comprising weighting the one or more trained machine learning models, wherein the score comprises one or more portions corresponding to the one or more trained machine learning models and the portions are proportional to the weights of the corresponding trained machine learning models, and further comprising:
receiving a correction associated with an outcome of the intervention; and
updating the weights of the one or more trained machine learning models based on the correction.

14. The system of claim 8, further comprising:
receiving feedback associated with the patient;
generating first processed patient information by identifying a first predictor variable and a first outcome variable in the patient information;
generating second processed patient information by identifying a second predictor variable and a second outcome variable in the feedback;
generating training data including:
the first information
the second information
the first processed patient information; and
the second processed patient information; and
training the one or more trained machine learning models using the training data to predict the first outcome variable and the second outcome variable using the first predictor variable and the second predictor variable.

15. A non-transitory computer-readable medium storing a set of instructions, the set of instructions comprising:
one or more instructions that, when executed by one or more processors of a device, cause the device to:
receive, from a first source, first information;
receive, from a second source, second information;
generate patient information by linking the first information and the second information using a linkage, corresponding to a patient;
generate, using one or more trained machine learning models, a risk prediction for the patient;
generate, using the one or more trained machine learning models, a change in risk prediction for the patient corresponding to an intervention; and
output the risk prediction and the change in risk prediction for the patient.

16. The non-transitory computer-readable medium of claim 15, further comprising receiving, from a third source, third information, wherein the third information includes engagement data from the patient.

17. The non-transitory computer-readable medium of claim 16, wherein the engagement data comprises a patient narrative and one or more social determinants of health.

18. The non-transitory computer-readable medium of claim 15, further comprising:
generating, using the one or more trained machine learning models, a score corresponding to the change in risk prediction for the patient, corresponding to the intervention, wherein the magnitude of the score corresponds to a likelihood of success associated with the intervention; and
outputting the score.

19. The non-transitory computer-readable medium of claim 18, further comprising weighting the one or more trained machine learning models, wherein the score comprises one or more portions corresponding to the one or more trained machine learning models and the portions are proportional to the weights of the corresponding trained machine learning models, and further comprising:
receiving a correction associated with an outcome of the intervention; and
updating the weights of the one or more trained machine learning models based on the correction.

20. The non-transitory computer-readable medium of claim 15, further comprising:
receiving feedback associated with the patient;
generating first processed patient information by identifying a first predictor variable and a first outcome variable in the patient information;
generating second processed patient information by identifying a second predictor variable and a second outcome variable in the feedback;
generating training data including:
the first information
the second information
the first processed patient information; and
the second processed patient information; and
training the one or more trained machine learning models using the training data to predict the first outcome variable and the second outcome variable using the first predictor variable and the second predictor variable.
